(19) United States
(12) Patent Application Publication
(10) Pub. No.: US 2003/0022871 A1
(43) Pub. Date: Jan. 30, 2003

(54) Method for modulating tumor growth and metastasis of tumor cells

(76) Inventor: Robert Benezra, Hampton Bays, NY (US)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 10/220,912

(22) PCT Filed: Mar. 8, 2001
(86) PCT No.: PCT/US01/07379

(51) Int. Cl. A61K 31/65; A61K 39/395

(52) U.S. Cl. 514/152

(57) ABSTRACT

Provided herein is a new and useful method for modulating, and particularly inhibiting angiogenesis in an animal. Since numerous diseases and disorders are dependent upon angiogenesis for continued growth in an animal, modulating and particularly inhibiting angiogenesis permits ultimately ameliorating a variety of diseases and disorders. Also provided are novel assays for screening compounds which may have applications in modulating and particularly inhibiting angiogenesis.

### FIELD OF THE INVENTION

The present invention relates generally to modulating the growth and metastasis of tumor cells, and particularly to shrinking tumors that have previously grown, and preventing growth of tumors by modulating the activity of the Id protein.

### BACKGROUND OF THE INVENTION

Cancer is considered to be a serious and pervasive disease. The National Cancer Institute has estimated that in the United States alone, 1 in 3 people will be struck with cancer during their lifetime. Moreover approximately 50% to 60% of people contracting cancer will eventually succumb to the disease. Hence, since the establishment the National Cancer Institute in the early 1970's, the amount of resources committed to cancer research has dramatically improved.

Although cancer is commonly considered to be a single disease, it actually comprises a family of diseases wherein normal cell differentiation is modified so that it becomes abnormal and uncontrolled. As a result, these malignant cells rapidly proliferate. Eventually, the cells spread or metastasize from their origin and colonize other organs, eventually killing their host. Due to the wide variety of cancers presently observed, numerous strategies have been developed to destroy cancer within the body. One such method utilizes cytotoxic chemotherapeutics. These compounds are administered to cancer suffers with the objective of destroying malignant cells while leaving normal, healthy cells undisturbed. Particular examples of such compounds include 5-fluorouracil, cisplatin, and methotrexate. However, chemotherapeutics also destroy normal, healthy cells, leaving the patient weakened and ill. Thus, chemotherapeutics possess inherent drawbacks.

Another strategy being studied for controlling cancer involves the use of signal transduction pathways in malignant cells to “turn off” their uncontrolled proliferation, or alternatively, instruct such cells to undergo apoptosis. Such methods of treating cancer are promising. However, a substantial amount of research is needed in order to make these methods viable alternatives.

Still another strategy involves cutting off the supply of oxygen and nutrients to the tumors so that they are unable to grow, and eventually die. Angiogenesis involves the formation of new blood vessels from existing blood vessels in response to various cell signals. Generally, new vessels form only when needed. However, it has been determined that angiogenesis also plays a role in the growth and metastasis of tumor cells. In particular, it has been determined that if a tumor was dependent upon an alternate method of supply, such as diffusion from surrounding tissue, it could not grow larger than approximately 1-2 millimeters in size. However, tumors grow to much larger sizes, and eventually spread to other parts of the body. In order to sustain its uncontrolled growth, endothelial cells and blood vessels promote the growth of a capillary network that invades the tumor mass. This network provides nutrients the tumor needs to grow, and permits the escape of metastatic cells from the tumor into the blood stream.

In 1990, a novel gene which encodes a novel helix-loop-helix (HLH) protein was isolated using a probe from the conserved region of amphipathic helix 2 [Benezra, R., Davis, R. L., Lockshon, D., Turner, D. L., and Weintraub, H. The protein Id: a negative regulator of helix-loop-helix DNA binding proteins. Cell 61:49-59 (1990)]. The HLH motif is a dimerization motif. After the protein dimerizes with another protein member of the HLH superfamily, the dimer binds to a DNA sequence in the major groove of DNA, and regulates expression of a particular gene. However, in order for DNA binding to occur, the dimer must form. Each protein of the dimer individually, is unable to regulate expression of a particular gene. Naturally, this novel HLH protein can associate and dimerize with other protein members of the HLH superfamily, including, but certainly not limited to Myo-D, E12, and E47, and attenuate their ability to bind specific DNA sequences and regulate the expression of particular genes.

Benezra et al. have also discovered this novel protein is distinct from other proteins of the HLH superfamily in that it lacks a basic region that is traditionally adjacent to the HLH domain. In addition, forced expression of the novel gene in transfection experiments was shown to inhibit Myo-D dependent activation of the MCK enhancer. Thus, the novel gene was labeled Id for “Inhibitor of DNA binding protein.” Naturally, the protein encoded by the Id gene is referred to as the Id protein.

It has also been determined that the Id gene is expressed in endothelial cells and blood vessels. In response to the Id protein, endothelial cells release chemical signals that promote angiogenesis, and permit infiltration of blood vessels into tumor growths. However, when blood vessels or endothelial cells are manipulated so that the Id gene is not expressed, angiogenesis is curtailed and blood vessels do not infiltrate tumor growths. Thus, the Id protein is associated with angiogenesis of blood vessels into tumors.

Accordingly, what is needed is a method of modulating the dimerization between an Id protein and another member of the HLH superfamily. As a result of this modulation, angiogenesis will be modulated.

### SUMMARY OF THE INVENTION

There is provided, in accordance with the present invention, a novel and useful method for modulating angiogenesis. As a result, blood vessels will not infiltrate a tumor growth, and continued growth and metastasis of the tumor will not be sustained.

Broadly, the present invention extends to a method of modulating angiogenesis in an animal comprising the administration of an effective amount of a compound that modulates dimerization between an Id protein and a protein member of the HLH superfamily.

The present invention further extends to a method of modulating angiogenesis as described above, wherein the compound interacts with the Id protein. As a result of this interaction, dimerization of the Id protein with a protein member of the HLH superfamily is modulated.

Furthermore, the present invention extends to a method of modulating angiogenesis as described above, wherein angiogenesis is inhibited and the compound's interaction with the Id protein prohibits the dimerization of the Id protein with a protein member of the HLH superfamily.

Numerous compounds have applications in a method of the invention. For example, the compound can be an antibody having an Id protein as an immunogen. Various types of antibodies, such as monoclonal, polyclonal and chimeric antibodies, have applications in the present invention.

Alternatively, the compound comprises an analog or derivative of tetracycline. Numerous analogs and derivatives of tetracycline have applications in a method of the invention. In a particular embodiment, an analog or derivative of tetracycline having applications herein has a general structure comprising:

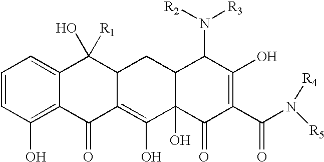

wherein R₁, R₂, R₃, R₄, and R₅ may be the same or different, and comprise H, lower alkyl (C₁-C₄), C₁-C₄ alkoxyl, cycloalkyl, aryl, or heterocyclic ring structures.

Other examples of analogs or derivatives of tetracycline having applications herein are set forth in U.S. Pat. Nos. 5,589,470; 5,064,821, 5,811,412; 4,089,900; 4,960,913; 4,066,694; 4,060,605; 3,911,111; and 3,951,962, which are hereby incorporated by reference herein in their entireties.

Naturally, a method of the present invention can modulate the dimerization activity of any presently known or subsequently discovered Id protein. Particular isoforms of the Id protein having applications here are the Id1, Id2, Id3, and Id4 proteins. The amino acid sequences of these proteins is readily available to the skilled artisan at GenBank with using a variety of databases, such as GENBANK, that are readily available to skilled artisans. Likewise, Id genes which encode these Id proteins encoded by these genes are also readily available.

Furthermore, the present invention extends to a method of modulating angiogenesis, and thus tumor growth and metastasis, as described above, wherein an effective amount of the compound inhibits expression of an Id gene. As a result of this inhibition, Id protein is no longer produced, and hence, dimerization between an Id protein and another protein member of the HLH superfamily is inhibited. Consequently, angiogenesis is inhibited.

Numerous compounds can be used to modulate expression of a particular gene, and have applications in the present invention. For example, modulating transcription of the gene and production of RNA modulates the gene's expression. Moreover, preventing translation of mRNA produced from the transcription of the gene also inhibits expression of the gene.

An antisense RNA compound described infra, which is designed to interact with Id mRNA, is an example of a compound that inhibits expression of the Id gene. Ribozymes, also described infra, can be constructed to digest Id mRNA, and thus prevent expression of the Id gene.

Naturally, a method of the invention can be used to modulate the expression of any allele of the Id gene presently known or subsequently discovered. Particular examples of alleles having applications herein includes, but certainly is no limited to Id1, Id2, Id3, and Id4 having nucleotide sequences set forth in SEQ ID NO: 1, SEQ ID NO:2, SEQ ID NO:3, and SEQ ID NO:4, respectively.

Moreover, any method of the present invention for modulating angiogenesis has ready applications in treating a variety of diseases and disorders related to angiogenesis, including, but not limited to a large number of cancers. Particular diseases and disorders that can be treated with a method of the present invention are set forth infra.

Furthermore, the present invention extends to a method of modulating tumor growth and metastasis in an animal, comprising administration to the animal of an effective amount of a compound that interacts with an Id protein, and modulates dimerization of the Id protein with a protein member of the HLH superfamily. A particular example of such a compound is an antibody having an Id protein as an immunogen. Particular antibodies having applications herein are described infra. Other examples of compounds having applications comprise analogs or derivatives of tetracycline. Generally, an analog or derivative of tetracycline having applications herein has a general structure comprising:

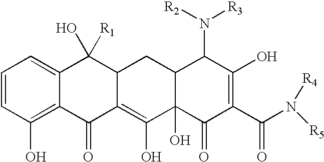

wherein R₁, R₂, R₃, R₄, and R₅ may be the same or different, and comprise H, lower alkyl (C₁-C₄), C₁-C₄ alkoxyl, cycloalkyl, aryl, or heterocyclic ring structures. Other examples of analogs or derivatives of tetracycline having applications herein are set forth in U.S. Pat. Nos. 5,589,470; 5,064,821, 5,811,412; 4,089,900; 4,960,913; 4,066,694; 4,060,605; 3,911,111; and 3,951,962. In a particular embodiment of a method of the invention, administration of an effective amount of the compound interacts with an Id protein and prevents its dimerization a protein member of the HLH superfamily. As result of this prevention of dimerization, angiogenesis is inhibited, and additional nutrients, oxygen, etc. can not be supplied to the tumor growth. As a result, the tumor growth and metastasis are inhibited. In a particular embodiment, methods of the present invention shrink tumors that had developed in the animal prior to administration of an effective amount of the compound.

A method of the present invention can be used prophylactically, e.g., prevent the development of any tumors or metastasis in the animal wherein the animal lacks any tumors prior to administration of an effective amount of the compound.

Naturally, as explained above, any Id protein presently known or subsequently discovered has applications in such a method of the invention. Particular examples include isoforms of the Id protein discussed above.

The present invention further extends to a method of modulating the activity of an Id protein comprising administration of an effective amount of a compound that binds to the Id protein and prevents its dimerization with a protein member of the HLH superfamily. When formation of the dimer is prevented, the dimer is not available to regulate genes involved in the angiogenesis process. Consequently, angiogenesis is prevented. Numerous compounds have applications in modulating the activity of the Id protein. Particular examples of such compounds are antibodies to an Id protein, analogs of tetracycline, or derivatives of tetracycline. Generally, analogs or derivatives of tetracycline having applications here have a general structure comprising:

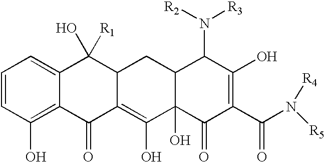

wherein R₁, R₂, R₃, R₄, and R₅ may be the same or different, and comprise H, lower alkyl (C₁-C₄), C₁-C₄ alkoxyl, cycloalkyl, aryl, or heterocyclic ring structures. Other examples of analogs or derivatives of tetracyclines that modulate the activity of the Id protein are set forth in U.S. Pat. Nos. 5,589,470; 5,064,821, 5,811,412; 4,089,900; 4,960,913; 4,066,694; 4,060,605; 3,911,111; and 3,951,962.

In another embodiment, the present invention extends to a method of using an Id protein in an assay for screening potential drugs or agents which interact with the Id protein, the method comprising the steps of:

a) providing the Id protein;

b) contacting the potential drug or agent to the Id protein; and

c) determining whether the potential drug or agent is bound to the Id protein.

A drug or agent that interacts with an Id protein may have applications in preventing the dimerization of the Id protein with a protein member of the HLH superfamily, and thus modulating angiogenesis. Consequently, such a drug or agent may have applications in modulating tumor growth and metastasis in an animal.

The present invention also extends to a method of using an Id protein in an assay for screening potential drugs or agents as described above, further comprising the steps of conjugating the Id protein to a solid phase resin prior to contacting the potential drug or agent to the Id protein, and removing the Id protein from the solid phase resin prior to determining whether the potential drug or agent is bound to the Id protein. Numerous solid phase resins have applications herein, e.g., cobalt, insoluble polystyrene beads, PVDF, and polyethylene glycol, to name only a few. A particular example of such a resin comprises a cobalt resin. Furthermore, when conjugating the Id protein to the resin, the protein to resin ratio must be sufficient to allow for saturation of the resin with the Id protein. One of ordinary skill in the art can readily determine an appropriate protein to resin ration using routine experimentation.

Likewise, numerous means are available for removing the Id protein from the resin after contact with the potential drug or agent. A particular example having applications herein comprises the Id protein conjugated to the solid phase resin with an imidazole solution.

Naturally, any Id protein presently known of subsequently discovered has applications in an assay of the invention. Particular examples of isoforms of the Id protein having applications herein are discussed above.

Furthermore, a potential drug or agent assayed with an assay of the invention can be a member of a library of compounds. Hence, the contacting step of an assay of the invention comprises contacting the library of compounds to the Id protein. Numerous libraries of compounds have applications herein. Particular examples comprise a mixture of compounds or a combinatorial library. In a particular embodiment, the library of compounds comprises analogs or derivatives of tetracycline.

In another embodiment, the present invention extends to a method of screening for compounds which selectively bind to an Id protein comprising:

(a) complexing the Id protein to a solid support;

(b) contacting the complexed protein/solid support with an aqueous solution comprising a compound that is being screened for the ability to selectively bind to the Id protein;

(c) determining whether the compound selectively binds to the Id protein such that the binding prevents dimerization of the Id protein with a protein member of the HLH superfamily.

Numerous solid supports have applications herein. Particular examples include, but certainly are not limited to cobalt, insoluble polystyrene beads, PVDF, and polyethylene glycol.

Naturally, animals which can be treated with a method of the present invention include those that are human, murine, bovine, ovine, porcine, feline, canine, and equine, to name only a few. What's more, an Id protein used in a method or assay of the invention can be obtained from any of these animals.

These and other aspects of the present invention will be better appreciated by reference to the following drawings and Detailed Description.

### BRIEF DESCRIPTION OF THE DRAWINGS

FIG. 1 is an autoradiograph of a native polyacrylamide gel used to determine that tetracycline interacts with a wild type Id protein.

FIG. 2 is an autoradiograph of a native polyacrylamide gel indicating that tetracycline modulates the dimerization of a wild type Id protein with a protein member of the HLH superfamily.

### DETAILED DESCRIPTION OF THE INVENTION

The present invention is based upon the discovery that surprisingly and unexpectedly, the activity of the Id protein is critical to the angiogenesis process. The Id protein is a member of the HLH superfamily. Thus, its activity comprises forming a dimer with another member of the HLH superfamily. This dimer then binds to DNA and regulates expression of genes involved in angiogenesis. Thus, modulating, and particularly preventing dimerization of the Id protein with another member of the HLH superfamily Id protein will modulate and particularly, prevent angiogenesis in the animal.

Broadly, the present invention extends to a method for modulating angiogenesis in an animal comprising the administration of an effective amount of a compound that modulates dimerization between an Id protein and a protein member of the HLH superfamily.

Numerous terms and phrases are used throughout the instant Specification and claims, and accordingly are defined below.

As used herein, the term “angiogenesis” refers to the growth of new blood vessels. Endothelial cells and blood vessels induce angiogenesis with secretion of chemical messengers. In the case of a malignancy, angiogenesis results in infiltration of capillaries into tumors. These capillaries supply nutrients and oxygen to the tumor to enable it to grow. They also form a conduit through which metastatic cells escape the tumor, enter the circulatory system, and metastasize in other organs of the body.

As used herein, the term “isoform” refers to multiple forms of the same protein that differ somewhat in their amino acid sequence. They can be produced by different genes or by alternative splicing of RNA transcripts from the same gene. Any isoform of the Id protein presently known or subsequently discovered has applications in the present invention. Particular isoforms having applications here include Id1, Id2, Id3, and Id4.

As used herein, the terms “modulating” or “modulation” refer to changing the rate at which a particular process occurs, such as angiogenesis, or alternatively to changing the activity of a compound, such as an Id gene. In a particular embodiment of the invention, “modulating” or “modulation” refer to inhibiting the dimerization of Id with a protein member of the HLH superfamily, inhibiting angiogenesis, and/or inhibiting tumor growth and metastasis.

As used herein, the phrase “effective amount” refers an amount sufficient to alter the rate of the angiogenesis by at least about 15 percent, preferably by at least 50 percent, more preferably by at least 90 percent, and most preferably prevent angiogenesis. As a result, tumor growths present in an animal prior to administration of an effective amount of compound are unable to continue to grow and metastasize, and preferably shrink in size. Alternatively, an “effective amount” of a compound administered to an animal is that amount sufficient to prevent formation of tumor growth in the animal, wherein the animal lacked any tumor formation prior to administration.

As used herein, the term “Id protein” refers to a protein produced from the expression of an Id gene. The phrase “Id gene” refers to a family of alleles, including Id1, Id2, Id3, or Id4. One of ordinary skill in the art can readily obtain nucleotide sequences for these Id genes using a variety of databases, such as GENBANK, that are readily available to skilled artisans. Likewise, the amino acid sequences of Id proteins encoded by these genes are also readily available.

As used herein, the terms “helix4oop-helix” and “HLH” can be used interchangeably, and refer to a secondary structure motif in polypeptides which comprises two 1-helices having the same orientation relative to each other and are connected by a loop region of similar structure, and is a DNA binding motif. Generally, in order to bind DNA, the HLH protein must dimerize with another HLH protein. Together, the HLH motifs form a unit that is responsible for differential binding to different operator DNA regions.

In addition, as set forth in the literature, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994), which are hereby incorporated by reference in their entireties, a “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced and translated into the protein encoded by the coding sequence.

A “signal sequence” is included at the beginning of the coding sequence of a protein to be expressed on the surface of a cell. This sequence encodes a signal peptide, N-terminal to the mature polypeptide, that directs the host cell to translocate the polypeptide. The term “translocation signal sequence” is used herein to refer to this sort of signal sequence. Translocation signal sequences can be found associated with a variety of proteins native to eukaryotes and prokaryotes, and are often functional in both types of organisms. An Id gene encoding an Id protein, whether genomic DNA or cDNA, can be isolated from any source, particularly from a human cDNA or genomic library. Methods for obtaining an Id gene are well known in the art (see, e.g., Sambrook et al., 1989, supra).

Accordingly, any animal cell potentially can serve as the nucleic acid source for the molecular cloning of an Id gene. The DNA may be obtained by standard procedures known in the art from cloned DNA (e.g., a DNA “library”), and preferably is obtained from a cDNA library prepared from tissues with high level expression of the protein, (e.g., tumor cell cDNA), by chemical synthesis, by cDNA cloning, or by the cloning of genomic DNA, or fragments thereof, purified from the desired cell (See, for example, Sambrook et al., 1989, supra; Glover, D. M. (ed.), 1985, DNA Cloning: A Practical Approach, MRL Press, Ltd., Oxford, U.K. Vol. I, II). Clones derived from genomic DNA may contain regulatory and intron DNA regions in addition to coding regions; clones derived from cDNA will not contain intron sequences. Whatever the source, the gene should be molecularly cloned into a suitable vector for propagation of the gene.

As explained above, the present invention is directed towards a method for modulating angiogenesis, and thus modulating the growth and metastasis of tumors, particularly solid tumors. As used herein, the terms “tumor” or “tumor growth” can be used interchangeably, and refer to an abnormal growth of tissue resulting from uncontrolled progressive multiplication of cells and serving no physiological function. A solid tumor can be malignant, e.g. tending to metastasize and being life threatening, or benign. Examples of solid tumors that can be treated according to a method of the present invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastorna, and retinoblastoma.

Moreover, tumors comprising dysproliferative changes (such as metaplasias and dysplasias) are treated or prevented in epithelial tissues such as those in the cervix, esophagus, and lung. Thus, the present invention provides for treatment of conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W. B. Saunders Co., Philadelphia, pp. 68-79). Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder. For a review of such disorders, see Fishman et al., 1985, Medicine, 2d Ed., J. B. Lippincott Co., Philadelphia.

Other examples of tumors that are benign and can be treated with a method of the present invention include arteriovenous (AV) malformations, particularly in intracranial sites and myoleomas. A method of the present invention may also be used to treat psoriasis, a dermatologic condition that is characterized by inflammation and vascular proliferation; benign prostatic hypertrophy, a condition associated with inflammation and possibly vascular proliferation; and cutaneous fungal infections. Treatment of other hyperprobiferative disorders is also contemplated.

### Methods for Modulating Angiogenesis

As explained throughout the instant specification, the present invention is directed towards, inter alia, modulating angiogenesis comprising the administration of an effective amount of a compound that interacts with an Id protein and prevents its dimerization with a protein member of the HLH superfamily. Interfering with formation of this dimer interferes with angiogenesis. Thus, tumor growth and metastasis, which is dependent upon angiogenesis, can be modulated. In practicing a method of the invention, a compound that interacts with an Id protein and prevents its dimerization with an HLH protein can be administered alone or in a composition comprising an the compound and a pharmaceutically acceptable carrier.

### Antibodies

According to the invention, a method for modulating angiogenesis comprises, inter alia administering to an animal an effective amount of an antibody having an Id protein as an immunogen. Such antibodies include but are not limited to polyclonal, monoclonal, chimeric, single chain, Fab fragments, and an Fab expression library. The anti-Id antibodies may be cross reactive, e.g., they may recognize Id protein from different species. Polyclonal antibodies have greater likelihood of cross reactivity. Alternatively, an antibody of the invention may be specific for a single form of Id such as murine, or a particular human isoform of the Id protein.

Various procedures known in the art may be used for the production of polyclonal antibodies to Id proteins. For the production of antibody, various host animals can be immunized by injection with an Id protein (e.g., fragment or fusion protein), including but not limited to rabbits, mice, rats, sheep, goats, etc. In one embodiment, the Id protein can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward an Id protein, any technique that provides for the production of antibody molecules by continuous cell lines in culture may be used. These include but are not limited to the hybridoma technique originally developed by Kohler and Milstein [Nature 256:495-497 (1975)], as well as the trioma technique, the human B-cell hybridoma technique [Kozbor et al., Immunology Today 4:72 1983); Cote et al., Proc. Natl. Acad. Sci. U.S.A. 80:2026-2030 (1983)], and the EBV-hybridoma technique to produce human monoclonal antibodies [Cole et al., in Monoclonal Antibodies and Cancer Therapy, Alan R. Liss, Inc., pp. 77-96 (1985)]. Monoclonal antibodies can also be produced in germ-free animals utilizing recent technology [PCT/US90/02545]. In fact, techniques developed for the production of “chimeric antibodies” [Morrison et al., J. Bacteriol. 159:870 (1984); Neuberger et al., Nature 312:604-608 (1984); Takeda et al., Nature 314:452454 (1985)] by splicing the genes from a mouse antibody molecule specific for an Id protein together with genes from a human antibody molecule of appropriate biological activity can be used; such antibodies have applications in a method of the present invention. Such human or humanized chimeric antibodies are preferred for use in therapy of human diseases or disorders, since the human or humanized antibodies are much less likely than xenogenic antibodies to induce an immune response, in particular an allergic response, themselves.

According to the invention, techniques described for the production of single chain antibodies [U.S. Pat. Nos. 5,476,786 and 5,132,405 to Huston; U.S. Pat. No. 4,946,778] can be adapted to produce Id protein-specific single chain antibodies. An additional embodiment of the invention utilizes the techniques described for the construction of Fab expression libraries [Huse et al., Science 246:1275-1281 (1989)] to allow rapid and easy identification of monoclonal Fab fragments with the desired specificity for an id protein.

Antibody fragments which contain the idiotype of the antibody molecule can be generated by known techniques. For example, such fragments include but are not limited to: the F(ab)₂ fragment which can be produced by pepsin digestion of the antibody molecule; the Fab fragments which can be generated by reducing the disulfide bridges of the F(ab)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of an Id protein, one may assay generated hybridomas for a product which binds to an Id protein containing such epitope. For selection of an antibody specific to an Id protein from a particular species of animal, one can select on the basis of positive binding with an Id protein expressed by or isolated from cells of that species of animal.

Suitable labels for detecting antibodies having applications here include enzymes, fluorophores (e.g., fluorescene isothiocyanate (FITC), phycoerythrin (PE), Texas red (TR), rhodamine, free or chelated lanthamide series salts, especially Eu³⁺, to name a few fluorophores), chromophores, radioisotopes, chelating agents, dyes, colloidal gold, latex particles, ligands (e.g., biotin), and chemiluminescent agents. When a control marker is employed, the same or different labels may be used for the receptor and control marker.

In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized colorimetric, spectrophotometric, fluorospectrophotometric, amperometric or gasometric techniques known in the art.

Direct labels are one example of labels that can be used according to the present invention. A direct label has been defined as an entity, which in its natural state, is readily visible, either to the naked eye, or with the aid of an optical filter and/or applied stimulation, e.g. U.V. light to promote fluorescence. Among examples of colored labels, which can be used according to the present invention, include metallic sol particles, for example, gold sol particles such as those described by Leuvering (U.S. Pat. No. 4,313,734); dye sol particles such as described by Gribnau et al. (U.S. Pat. No. 4,373,932) and May et al. (WO 88/08534); dyed latex such as described by May, supra, Snyder (EP-A 0 280 559 and 0 281 327); or dyes encapsulated in liposomes as described by Campbell et al. (U.S. Pat. No. 4,703,017). Other direct labels include a radionucleotide, a fluorescent moiety or a luminescent moiety. In addition to these direct labeling devices, indirect labels comprising enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, urease, these and others have been discussed in detail by Eva Engvall in Enzyme Immunoassay ELISA and EMIT in Methods in Enzymology, 70. 419439, 1980 and in U.S. Pat. No. 4,857,453.

Other labels for use in the invention include magnetic beads or magnetic resonance imaging labels.

In addition, a phosphorylation site can be created on an antibody with ³²P, e.g., as described in European Patent No. 0372707 (application No. 89311108.8) by Sidney Pestka, or U.S. Pat. No. 5,459,240, issued Oct. 17, 1995 to Foxwell et al.

As exemplified herein, proteins, including antibodies, can be labeled by metabolic labeling. Metabolic labeling occurs during in vitro incubation of the cells that express the protein in the presence of culture medium supplemented with a metabolic label, such as [³⁵S]-methionine or [³²P]-orthphosphate. In addition to metabolic (or biosynthetic) labeling with [³⁵S]-methionine, the invention further contemplates labeling with [¹⁴C]-amino acids and [³H]-amino acids (with the tritium substituted at non-labile positions).

The foregoing antibodies can be used in methods known in the art relating to the localization and activity of an Id protein, e.g., for Western blotting, imaging an Id protein in situ, measuring levels thereof in appropriate physiological samples, etc. using any of the detection techniques mentioned above or known in the art.

Naturally, antibodies having applications herein modulate dimerization between Id and a protein member of the HLH superfamily. Thus, such antibodies modulate angiogenesis, tumor growth and metastasis, etc. Such antibodies can be tested using assays described infra.

### Antisense Compounds

The present invention extends to the preparation of antisense nucleic acid molecules and ribozymes that may be used to modulate, and particularly, inhibit expression of an Id gene at the translational level. Obviously, modulating expression of an Id gene modulates the availability of Id protein, and thus, dimerization of an Id protein with another protein member of the HLH superfamily.

In particular, this approach utilizes antisense nucleic acid and ribozymes to block translation of a specific mRNA, either by masking that mRNA with an antisense nucleic acid or cleaving it with a ribozyme.

Antisense nucleic acids are DNA or RNA molecules that are complementary to at least a portion of a specific mRNA molecule [see Marcus-Sekura, Anal. Biochem. 172:298 (1988)]. In the cell, they hybridize to that mRNA, forming a double stranded molecule. The cell does not translate an mRNA in this double-stranded form. Therefore, antisense nucleic acids interfere with the expression of mRNA into protein. Oligomers of about fifteen nucleotides and molecules that hybridize to the AUG initiation codon will be particularly efficient, since they are easy to synthesize and are likely to pose fewer problems than larger molecules when introducing them into organ cells. Antisense methods have been used to inhibit the expression of many genes in vitro [Marcus-Sekura, 1988, supra; Hambor et al., J. Exp. Med 168:1237 (1988)]. Preferably synthetic antisense nucleotides contain phosphoester analogs, such as phosphorothiolates, or thioesters, rather than natural phophoester bonds. Such phosphoester bond analogs are more resistant to degradation, increasing the stability, and therefore the efficacy, of the antisense nucleic acids.

Ribozymes are RNA molecules possessing the ability to specifically cleave other single stranded RNA molecules in a manner somewhat analogous to DNA restriction endonucleases. Ribozymes were discovered from the observation that certain mRNAs have the ability to excise their own introns. By modifying the nucleotide sequence of these RNAs, researchers have been able to engineer molecules that recognize specific nucleotide sequences in an RNA molecule and cleave it [Cech, J. Am. Med. Assoc. 260:3030 (1988)]. Because they are sequence-specific, only mRNAs with particular sequences are inactivated.

Investigators have identified two types of ribozymes, Tetrahymena-type and “hammerhead”-type. Tetrahymena-type ribozymes recognize four-base sequences, while “hammerhead”-type recognize eleven- to eighteen-base sequences. The longer the recognition sequence, the more likely it is to occur exclusively in the target mRNA species. Therefore, hammerhead-type ribozymes are preferable to Tetrahymena-type ribozymes for inactivating a specific mRNA species, and eighteen base recognition sequences are preferable to shorter recognition sequences.

The DNA sequences encoding Id proteins described herein may thus be used to prepare antisense molecules against and ribozymes that cleave mRNAs for the Id protein, thus inhibiting expression of the gene encoding the Id protein. As a result, Id protein is not produced and naturally, dimerization between Id and another member of the HLH superfamily is modulated to the point of not occurring.

Analogs or Derivatives of Tetracycline

In addition, the present invention extends to methods for modulating angiogenesis in an animal comprising, inter alia, administration of an effective amount of an analog or derivative of tetracycline. As used herein, “tetracycline” refers to a compound having an elemental formula of C₂₂H₂₄N₂O8 and nomenclature of [4S-(4I,5aI,5aI,6J,12aI)]-4-(Dimethylamino)-1,4,4a,5,5a,6-11,12a-octahydro-3,6, 10,12,12a-peiztaiydroxy-6-methyl-1,11-dioxo-2-naphthacenecarboxamide. The structure of tetracycline is set forth below:

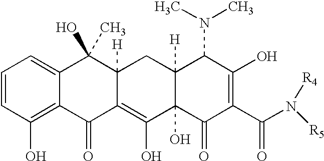

An analog or derivative of tetracycline refers to a chemical compound derived or obtained from tetracycline and containing essential elements of tetracycline. Generally, an analog or derivative of tetracycline having applications herein comprises a general structure of:

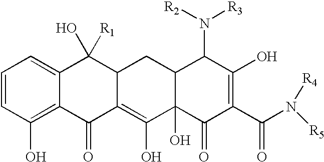

wherein R₁, R₂, R₃, R₄, and R₅ may be the same or different, and comprise H, lower alkyl (C₁-C₄), C₁-C₄ alkoxyl, cycloalkyl, aryl, or heterocyclic ring structures. Other examples of analogs or derivatives of tetracyclines that modulate the activity of the Id protein are set forth in U.S. Pat. Nos. 5,589,470; 5,064,821, 5,811,412; 4,089,900; 4,960,913; 4,066,694; 4,060,605; 3,911,111; and 3,951,962.

### Administration of a Compound that Modulates Angiogenesis

As explained above, the present invention extends a method for modulating angiogenesis in an animal comprising administration of an effective amount of a compound that modulates dimerization between an Id protein and a protein member of the HLH superfamily.

An “effective amount” refers to an amount sufficient to alter, and particularly decrease the rate of the angiogenesis by at least about 15 percent, preferably by at least 50 percent, more preferably by at least 90 percent, and most preferably prevent angiogenesis. As a result, tumor growths present in an animal prior to administration of an effective amount of compound are unable to continue to grow and metastasize, and preferably shrink in size. Alternatively, an “effective amount” of a compound administered to an animal is that amount sufficient to prevent formation of tumor growth in the animal, wherein the animal lacks any tumor formation prior to administration. Thus, an effective amount can be prophylactic. Generally, compounds can be administered alone or in a pharmaceutical composition comprising a compound that modulates angiogenesis and a pharmaceutically acceptable carrier thereof. The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

Moreover, numerous means for administrating a compound which modulates dimerization of an Id protein and a member of the HLH superfamily have applications in a method of the present invention. In particular, a therapeutic composition comprising a compound that modulates such dimerization may be introduced parenterally, transmucosally, e.g., orally, nasally, or rectally, or transdermally. Preferably, administration is parenteral, e.g., via intravenous injection, and also including, but is not limited to, intra-arteriole, intramuscular, intradermal, subcutaneous, intraperitoneal, intraventricular, and intracranial administration. More preferably, it may be introduced by injection into the tumor(s) being treated or into tissues surrounding the tumor(s).

In another embodiment, according to a method of the present invention, a composition comprising a compound that modulates Id dimerization to another member of the HLH superfamily can delivered in a vesicle, in particular a liposome [see Langer, Science 249:1527-1533 (1990); Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.].

In yet another embodiment, such a compound can be delivered in a controlled release system. For example, a compound that interacts with an Id protein so that it can not dimerize with another HLH protein can be administered using intravenous infusion, an implantable osmotic pump, a transdermal patch, liposomes, or other modes of administration. In a particular embodiment, a pump may be used [see Langer, supra; Sefton, CRC Crit. Ref. Biomed. Eng. 14:201 (1987); Buchwald et al., Surgery 88:507 (1980); Saudek et al., N. Engl. J. Med 321:574 (1989)]. In another embodiment, polymeric materials can be used [see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)]. In yet another embodiment, a controlled release system can be placed in proximity of the target, i.e., the brain, thus requiring only a fraction of the systemic dose [see, e.g., Goodson, in Medical Applications of Controlled Release, supra, vol. 2, pp. 115-138 (1984)]. Preferably, a controlled release device is introduced into an animal in proximity of the site of inappropriate immune activation or a tumor. Other controlled release systems are discussed in the review by Langer [Science 249:1527-1533 (1990)].

### Nasal Delivery

Nasal delivery of a compound that modulates Id dimerization with another member of the HLH superfamily is also contemplated. Nasal delivery allows the passage of such a compound to the blood stream directly after administering an effective amount of the compound to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition into a chamber of defined volume, which chamber has an aperture dimensioned to aerosolize and aerosol formulation by forming a spray when a liquid in the chamber is compressed. The chamber is compressed to administer the pharmaceutical composition comprising a compound that modulates dimerization of an Id protein with another HLH protein. In a specific embodiment, the chamber is a piston arrangement. Such devices are commercially available.

Alternatively, a plastic squeeze bottle having an aperture or opening dimensioned to aerosolize an aerosol formulation can be used. Aerolsolization occurs when the bottle is squeezed. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

### Oral Delivery

Contemplated for use herein are oral solid dosage forms, which are described generally in Remington's Pharmaceutical Sciences, 18th Ed. 1990 (Mack Publishing Co. Easton Pa. 18042) at Chapter 89, which is herein incorporated by reference. Solid dosage forms include tablets, capsules, pills, troches or lozenges, cachets or pellets. Also, liposomal or proteinoid encapsulation may be used to formulate the compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given by Marshall, K. In: Modern Pharmaceutics Edited by G. S. Banker and C. T. Rhodes Chapter 10, 1979, herein incorporated by reference. In general, the formulation will include a compound that modulates Id dimerization with another protein of the HLH superfamily.

Also specifically contemplated are oral dosage forms of a composition comprising a compound that Id dimerization in a method of the present invention. Such a compound may be chemically modified so that oral delivery is efficacious. Generally, the chemical modification contemplated is the attachment to the compound of at least one moiety that permits (a) inhibition of proteolysis; and (b) uptake into the blood stream from the stomach or intestine. Also desired is the increase in overall stability of the compound and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-trioxocane. Preferred for pharmaceutical usage, as indicated above, are polyethylene glycol moieties.

In a method of the present invention, the location of release of the compound may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound or by release of the compound beyond the stomach environment, such as in the intestine. To ensure full gastric resistance, a coating should be impermeable to at least pH 5.0. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

A compound modulates Id dimerization with another HLH protein can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, a compound which interacts with an Id protein and modulates its dimerization with another HLH protein may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, I-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include, but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold a composition comprising a compound that modulates Id dimerization to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticling during the formulation process. Lubricants may be used as a layer between the therapeutic and the die wall, and these can include but are not limited to: stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of a compound that modulates Id dimerization with another HLH protein in the aqueous environment, a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the protein or derivative either alone or as a mixture in different ratios.

Additives such as the fatty acids oleic acid, linoleic acid and linolenic acid, potentially enhance uptake of a compound that interacts with an Id protein and modulates its dimerization with another HLH protein.

Controlled release oral formulation may be desirable. The drug could be incorporated into an inert matrix which permits release by either diffusion or leaching mechanisms, e.g., gums. Slowly degenerating matrices may also be incorporated into the formulation. Some enteric coatings also have a delayed release effect.

Another form of a controlled release of this therapeutic is by a method based on the Oros therapeutic system (Alza Corp.), i.e. the drug is enclosed in a semipermeable membrane which allows water to enter and push drug out through a single small opening due to osmotic effects.

Other coatings may be used for the formulation. These include a variety of sugars which could be applied in a coating pan. The therapeutic agent could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide the optimum film coating. Film coating may be carried out in a pan-coater or in a fluidized bed or by compression coating.

### Pulmonary Delivery

Also contemplated for use in a method of the present invention is pulmonary delivery. A compound that modulates Id dimerization with another HLH protein can be delivered to the lungs of an animal while inhaling and traverses across the lung epithelial lining to the blood stream. Other reports of this include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J. Immunol. 140:3482-3488 (interferon-K and tumor necrosis factor-I) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of the invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colorado; the Ventolin metered dose inhaler, manufactured by Glaxo Inc., Research Triangle Park, North Carolina; and the Spinhaler powder inhaler, manufactured by Fisons Corp., Bedford, Mass.

All such devices require the use of formulations suitable for the dispensing of a compound that modulates dimerization of an Id protein with another protein member of the HLH superfamily. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified Id protein may also be prepared in different formulations depending on the type of chemical modification or the type of device employed.

Formulations suitable for use with a nebulizer, either jet or ultrasonic, will typically comprise compound(s) that modulate dimerization of an Id protein and another protein member of the HLH superfamily dissolved in water at a concentration of about 0.1 to 25 mg of compound(s) per mL of solution. The formulation may also include a buffer and a simple sugar (e.g., for compound stabilization and regulation of osmotic pressure). The nebulizer formulation may also contain a surfactant to reduce or prevent surface induced aggregation of the protein caused by atomization of the solution in forming the aerosol. Formulations for use with a metered-dose inhaler device will generally comprise a finely divided powder containing a compound that modulates Id dimerization suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing protein (or derivative) and may also include a bulking agent, such as lactose, sorbitol, sucrose, or mannitol in amounts which facilitate dispersal of the powder from the device, e.g., 50 to 90% by weight of the formulation. The compound to be administered should most advantageously be prepared in particulate form with an average particle size of less than 10 mm (or microns), most preferably 0.5 to 5 mm, for most effective delivery to the distal lung.

### Transdermal Administration

Various and numerous methods are known in the art for transdermal administration of a compound that modulate dimerization of an Id protein with another protein member of the HLH superfamily, e.g., via a transdermal patch. Transdermal patches are described in for example, U.S. Pat. No. 5,407,713, issued Apr. 18, 1995 to Rolando et al.; U.S. Pat. No. 5,352,456, issued Oct. 4, 1004 to Fallon et al.; U.S. Pat. No. 5,332,213 issued Aug. 9, 1994 to D'Angelo et al.; U.S. Pat. No. 5,336,168, issued Aug. 9, 1994 to Sibalis; U.S. Pat. No. 5,290,561, issued Mar. 1, 1994 to Farhadieh et al.; U.S. Pat. No. 5,254,346, issued Oct. 19, 1993 to Tucker et al.; U.S. Pat. No. 5,164,189, issued Nov. 17, 1992 to Berger et al.; U.S. Pat. No. 5,163,899, issued Nov. 17, 1992 to Sibalis; U.S. Pat. Nos. 5,088,977 and 5,087,240, both issued Feb. 18, 1992 to Sibalis; U.S. Pat. No. 5,008,110, issued Apr. 16, 1991 to Benecke et al.; and U.S. Pat. No. 4,921,475, issued May 1, 1990 to Sibalis, the disclosure of each of which is incorporated herein by reference in its entirety.

It can be readily appreciated that a transdermal route of administration may be enhanced by use of a dermal penetration enhancer, e.g., such as enhancers described in U.S. Pat. No. 5,164,189 (supra), U.S. Pat. No. 5,008,110 (supra), and U.S. Pat. No. 4,879,119, issued Nov. 7, 1989 to Aruga et al., the disclosure of each of which is incorporated herein by reference in its entirety.

### Assays for Compounds that Modulate Id Protein Activity

As explained above, the present invention extends to, inter alia, an assay for potential drugs or agents which modulate Id protein activity, i.e., dimerization of an Id protein with a member of the HLH superfamily, and particularly, prevent the Id protein from dimerizing with a member of the HLH superfamily. As a result, the drug or agent may have potential as a therapeutic for modulating tumor growth and metastasis.

Naturally, an assay of the invention comprises, inter alia, contacting the Id protein with the potential drug or agent. Optionally, such contact can occur in solution, e.g., TRIS buffer, or phosphate buffered saline (PBS) at physiological pH.

Alternatively, an assay of the present invention involves, inter alia, conjugating an Id protein to a solid support. Solid supports having applications in such an assay of the invention include membranes (e.g., nitrocellulose or nylon), a microtiter dish (e.g., PVC, polypropylene, or polystyrene), a test tube (glass or plastic), a dip stick (e.g., glass, PVC, polypropylene, polystyrene, latex and the like), a microfuge tube, or a glass, silica, plastic, metallic or polymer bead or other substrate such as paper. A particular solid support having applications in an assay of the invention comprises a cobalt or nickel column which binds with specificity to a histidine tag engineered onto an Id protein using routine molecular biology techniques well known to those of ordinary skill in the art.

Adhesion of the Id protein to the solid support can be direct (i.e. the protein contacts the solid support) or indirect (a particular compound or compounds are bound to the support and the target protein binds to this compound rather than the solid support). One can immobilize Id proteins either covalently (e.g., utilizing single reactive thiol groups of cysteine residues (see, e.g., Colliuod et al. Bioconjugate Chem. 4:528-536 (1993)) or non-covalently but specifically (e.g., via immobilized antibodies (Schubmann et al. Adv. Mater. 3:388-391 (1991); Lu et al. Anal. Chem. 67:83-87 (1995), the biotin/strepavidin system (Iwane et al. Biophys. Biochem. Res. Comm. 230:76-80 (1997) or metal chelating Langmuir-Blodgett films (Ng et al. Langmuir 11:4048-55 (1995); Schmitt et al. Angew. Chem. Int. Ed. Engl. 35:317-20 (1996); Frey et al. Proc. Natl. Acad. Sci. USA 93:493741 (1996); Kubalek et al. J. Struct. Biol. 113:117-123 (1994)) and metal-chelating self-assembled monolayers (Sigal et al. Anal. Chem. 68:490497 (1996)) for binding of polyhistidine fusions.

Indirect binding can be achieved using a variety of linkers which are commercially available. The reactive ends can be any of a variety of functionalities including, but not limited to: amino reacting ends such as N-hydroxysuccinimide (NHS) active esters, imidoesters, aldehydes, epoxides, sulfonyl halides, isocyanate, isothiocyanate, and nitroaryl halides; and thiol reacting ends such as pyridyl disulfides, maleimides, thiophthalimides, and active halogens. The heterobifunctional crosslinking reagents have two different reactive ends, e.g., an amino-reactive end and a thiol-reactive end, while homobifunctional reagents have two similar reactive ends, e.g., bismaleimidohexane (BMH) which permits the cross-linking of sulfhydryl-containing compounds. The spacer can be of varying length and be aliphatic or aromatic. Examples of commercially available homobifunctional cross-linking reagents include, but are certainly not limited to the imidoesters such as dimethyl adipimidate dihydrochloride (DMA); dimethyl pimelimidate dihydrochloride (DMP); and dimethyl suberimidate dihydrochloride (DMS).

Heterobifunctional reagents include commercially available active halogen-NHS active esters coupling agents such as N-succinimidyl bromoacetate and N-succinimidyl(4-iodoacetyl)aniinobenzoate (SIAB) and the sulfosuccinimidyl derivatives such as sulfosuccinimidyl(4-iodoacetyl)aminobenzoate (sulfo-SIAB) (Pierce). Another group of coupling agents is the heterobifunctional and thiol cleavable agents such as N-succinimidyl 3-(2-pyridyidithio)propionate (SPDP) (Pierce).

Antibodies are also available for binding Id proteins to a solid support. This can be done directly by binding Id protein specific antibodies to the column and allowing Id proteins to bind or it can be done by creating chimeras constructed from Id proteins linked to an appropriate immunoglobulin constant domain sequence. Such species are referred to as immunoadhesins, and are known to those of ordinary skill in the art. Immunoadhesins reported in the literature include Gascoigne et al., Proc. Natl. Acad. Sci. USA 84,. 2936-2940 (1987), Capon et al., Nature 377, 525-531 (1989); and Traunecker et al., Nature 33, 68-70 (1989).

By manipulating the solid support and the mode of attachment of the target molecule to the support, it is possible to control the orientation of the target molecule. Thus, for example, where it is desirable to attach a target molecule to a surface in a manner that leaves the HLH motif of the Id protein to interact with other molecules, a tag (e.g., FLAG, myc, GST, polyHis, etc.) may be added to the target molecule at a particular position in the target sequence.

### Assays

Once bound, there are a variety of assay formats that can be used to screen for modulators of dimerization between an Id protein and another member of the HLH superfamily. Various molecules that interact with an Id protein can be identified by (1) attaching the Id protein (“the target”) to a solid support, (2) contacting a second molecule with the support coated with the Id protein, and 3) detecting the binding of the second molecule to the Id protein. Molecules that interact or bind with the target are then eluted, with or without the target, thereby isolating molecules that interact with the target.

For a general description of different formats for binding assays, see BASIC AND CLINICAL IMMUNOLOGY, 7th Ed. (D. Stiles and A. Terr, ed.)(1991); ENZYME IMMUNOASSAY, E. T. Maggio, ed., CRC Press, Boca Raton, Fla. (1980); and “Practice and Theory of Enzyme Immunoassays” in P. Tijssen, LABORATORY TECHNIQUES IN BIOCHEMISTRY AND MOLECULAR BIOLOGY, Elsevier Science Publishers, B.V. Amsterdam (1985), each of which is incorporated by reference.

In competitive binding assays, the test compound competes with a second compound for specific binding sites on a target molecule attached to the solid support. Binding is determined by assessing the amount of second compound associated with the target molecule. The amount of second compound associated with the target molecule is inversely proportional to the ability of a test compound to compete in the binding assay. The amount of inhibition or stimulation of binding of a labeled target by the test compound depends on the binding assay conditions and on the concentrations of binding agent, labeled analyte and test compounds used. Under specified assay conditions, a compound is said to be capable of inhibiting the binding of a second compound to a target compound if the amount of bound second compound is decreased by 50% or preferably 90% or more compared to a control sample.

Alternatively, various known or unknown compounds, including proteins, carbohydrates, and the like, can be assayed for their ability to interact with an Id protein. In one embodiment, samples from various tissues are contacted with the target to isolate molecules that interact with the target. In another embodiment, small molecule libraries and high throughput screening methods are used to identify compounds that bind to the target.

### Labels for Use in Assays

The amount of binding of the second compound to a target Id protein can be assessed by directly labeling the second compound with a detectable moiety, or by detecting the binding of a labeled ligand that specifically binds to the second compound. A wide variety of labels can be used. The detectable labels of the invention can be primary labels (where the label comprises an element that is detected or that produces a directly detectable signal) or secondary labels (where the detected label binds to a primary label, e.g., as is common in immunological labeling). An introduction to labels, labeling procedures and detection of labels is found in Polak and Van Noorden (1997) Introduction to Immunochemistry, 2nd ed., Springer Verlag, NY and in Haugland (1996) Handbook of Fluorescent Probes and Research Chemicals, a combined catalog and handbook published by Molecular Probes, Inc., Eugene, Oreg. Useful primary and secondary labels having applications in an assay of the present invention include spectral labels such as fluorescein isothiocyanate (FITC) and Oregon Green, rhodamine and derivatives (e.g. Texas red, tetrarhodimine isothiocyanate (TRITC), etc.), digoxigenin, biotin, phycoerythrin, AMCA, CyDyes, and the like), radiolabels (e.g., ³H, ¹²⁵I, ³⁵S, ¹⁴C or ³²P), enzymes (e.g. horseradish peroxidase, alkaline phosphatase, etc.), spectral colorimetric labels such as colloidal gold and colored glass or plastic (e.g. polysytrene, polypropylene. latex, etc.) beads, to name only a few. The choice of label depends on sensitivity required, ease of conjugation with the compound, stability requirements, and available instrumentation.

In general, a detector that monitors a particular probe or probe combination is used to detect the recognition reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill.

### High-Throughput Screening of Candidate Agents that Modulate Activity of Id Proteins

Conventionally, new chemical entities with useful properties are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one embodiment of an assay of the invention, high throughput screening methods involve providing a library containing a large number of potential therapeutic compounds (candidate compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

### Combinatorial Chemical Libraries

Combinatorial chemical libraries are a preferred means to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 12331250).

Preparation and screening of combinatorial chemical libraries are understood by those of ordinary skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, Dec. 26, 1991), encoded peptides (PCT Publication WO 93/20242, Oct. 14, 1993), random biooligomers (PCT Publication WO 92/00091, Jan. 9, 1992), benzodiazepines (U.S. Pat. No. 5,288,514), diversomers such as hydantoins, benzodiazepines and dipeptides (Hobbs et al., (1993) Proc. Nat. Acad. Sci. USA 90: 69096913), vinylogous polypeptides (Hagihara et al. (1992) J. Amer. Chem. Soc. 114: 6568), nonpeptidal peptidomimetics with a Beta D Glucose scaffolding (Hirschmann et al., (1992) J. Amer. Chem. Soc. 114: 92179218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries, peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, Jan 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines U.S. Pat. No. 5,288,514, and the like). In a particular embodiment of an assay of the invention, such a library comprises a large variety of analogs or derivatives of tetracycline.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, HewlettPackard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, MD, etc.).

### High Throughput Assays of Chemical Libraries

Any of the assays for compounds capable of modulating the dimerization of an Id protein with a protein member of the HLH superfamily are amenable to high throughput screening. High throughput screening systems are commercially available (see, e.g., Zymark Corp., Hopkinton, Mass.; Air Technical Industries, Mentor, Ohio; Beclaan Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high thruput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like. Particle methods of determining whether a molecule has interacted with an Id protein are described infra.

### Analogs and Derivatives of Tetracycline

As explained above, the present invention is directed towards, inter alia, a method of modulating, and particularly, inhibiting tumor growth and metastasis comprising administration of an effective amount of a compound that interacts with an Id protein, and modulates its dimerization with another protein member of the HLH superfamily. Particular examples of compounds having applications herein comprise analogs or derivatives of tetracycline. Numerous analogs and derivatives are readily available to the skilled artisan. Particular examples of analogs or derivatives having applications herein include, but certainly are not limited to a general formula of:

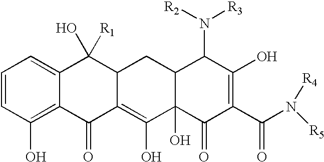

wherein R₁, R₂, R₃, R₄, and R₅ may be the same or different, and comprise H, lower alkyl (C₁-C₄), C₁-C₄ alkoxyl, cycloalkyl, aryl, or heterocyclic ring structures. These and other analogs and derivatives of tetracycline can be readily prepared by one of ordinary skill in the art using routine experimental techniques.

In addition, tetracycline can also be derivatized by the attachment of one or more chemical moieties to tetracycline. The chemically modified derivatives may be further formulated for intraarterial, intraperitoneal, intramuscular subcutaneous, intravenous, oral, nasal, pulmonary, topical or other routes of administration. Chemical modification of tetracycline may provide additional advantages under certain circumstances, such as increasing the stability and circulation time of the analog or derivative of tetracycline. See U.S. Pat. No. 4,179,337, Davis et al., issued Dec. 18, 1979. For a review, see Abuchowski et al., in Enzymes as Drugs (J. S. Holcerberg and J. Roberts, eds. pp. 367-383 (1981)).

Chemical Moieties For Derivatization. The chemical moieties suitable for derivatization may be selected from among water soluble polymers. The polymer selected should be water soluble so that the component to which it is attached does not precipitate in an aqueous environment, such as a physiological environment. Preferably, for therapeutic use of the end-product preparation, the polymer will be pharmaceutically acceptable. One skilled in the art will be able to select the desired polymer based on such considerations as whether the polymer/component conjugate will be used therapeutically, and if so, the desired dosage, circulation time, resistance to proteolysis, and other considerations. For analogs or derivatives of tetracycline, these may be ascertained using the assays provided herein.

The water soluble polymer may be selected from the group consisting of, for example, polyethylene glycol, copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, glycol homopolymers, polypropylene oxide/ethylene oxide co-polymers, polyoxyethylated polyols and polyvinyl alcohol. Polyethylene glycol propionaldenhyde may have advantages in manufacturing due to its stability in water.

The polymer may be of any molecular weight, and may be branched or unbranched. For polyethylene glycol, the preferred molecular weight is between about 2 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The number of polymer molecules so attached may vary, and one skilled in the art will be able to ascertain the effect on function. One may mono-derivatize, or may provide for a di-, tri-, tetra- or some combination of derivatization, with the same or different chemical moieties (e.g., polymers, such as different weights of polyethylene glycols). The proportion of polymer molecules to an analog or derivative of tetracycline will vary, as will their concentrations in the reaction mixture. In general, the optimum ratio (in terms of efficiency of reaction in that there is no excess unreacted component or components and polymer) will be determined by factors such as the desired degree of derivatization (e.g., mono, di-, tri-, etc.), the molecular weight of the polymer selected, whether the polymer is branched or unbranched, and the reaction conditions.

Other analogs or derivatives of tetracycline having applications in a method of the present invention are set forth in U.S. Pat. Nos. 5,589,470; 5,064,821, 5,811,412; 4,089,900; 4,960,913; 4,066,694; 4,060,605; 3,911,111; and 3,951,962, which are hereby incorporated by reference herein in their entireties.

The present invention may be better understood by reference to the following non-limiting Examples, which are provided as exemplary of the invention. The following examples are presented in order to more fully illustrate the preferred embodiments of the invention. They should in no way be construed, however, as limiting the broad scope of the invention.

### EXAMPLES

### Example I

### Demonstration that Tetracycline Binds to Native Id1 Protein

General Strategy:

Radiolabeled tetracycline (tritiated) was mixed with purified Id1 protein and other proteins separately to permit contact between the labeled tetracycline and the proteins. Each mixture protein and labeled tetracycline was then electrophoresed on a native (non-denaturing) polyacrylamide gel to separate free tetracycline (which does not run into the gel) from any complexes that may have formed between tetracycline and the respective proteins. The complexes could then be visualized in an autoradiogram of the gel, e.g., exposure of the gel to x-ray film.

In this experiment, a 10-30-fold molar excess of tritiated tetracycline was added to the indicated proteins separately in 10 Tl reaction tube containing one of the proteins and phosphate buffered saline at physiological pH. Proteins used in this experiment were:

(1) Hsp90 (heat shock protein 90);

(2) E12, a member of the HLH protein superfamily which dimerizes with Id;

(3) Id old, an old preparation of Id in which the protein's conformation is perturbed;

(4) Id1 new, the normal protein;

(5) MyoD, another member of the HLH protein superfamily; and

(6) E47, another member of the HLH protein superfamily that interacts with Id.

The mixtures were incubated at room temperature for 30 minutes. Bromophenol blue was added to each sample at 0.01% (v/v). The samples were then electrophoresed on an 8% native polyacrylamide gel.

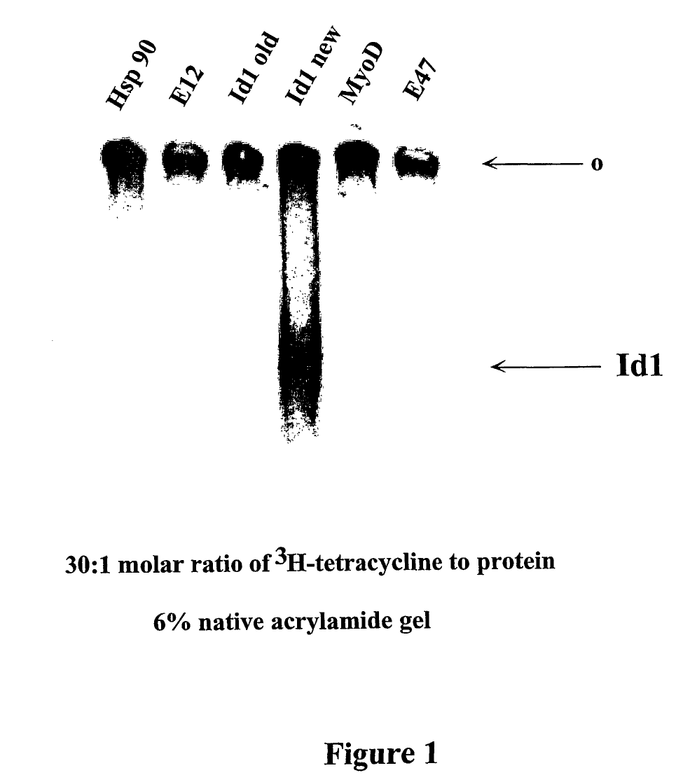

After electrophoresing the samples, the gel was dried and exposed to autoradiographic film for 12 hours to make an autoradiogram of the gel. A photograph of the radiogram is set forth in FIG. 1.

Results:

No radiolabeled tetracycline was detected in lanes 1-3, and 5-6. Thus, tetracycline did not bind to respective proteins of these lanes. Moreover, no radiolabeled tetracycline was detected in lane 3, which contained old Id having a perturbed conformation. However, labeled tetracycline was detected in lane 4, which contained native Id1 protein. Since, as explained above, tetracycline does not run into polyacrylamide gels, Such detection could only occur if the labeled tetracycline bound to native Id1 during the incubation of the Id1 sample with labeled tetracycline. Thus, tetracycline, along with analogs or derivatives thereof, binds to native Id protein.

### Example II

### Demonstration that Tetracycline Inhibits the Activity of Id1 in vitro

General Strategy:

Purified Id1 and its target protein E12 are mixed to together in vitro, and the ability of Id1 to antagonize DNA binding capacity of E12 is measured. To measure E12 DNA binding activity, the mixtures were incubated with radiolabeled DNA sequences to which E12 binds and the protein/DNA complexes resolved on a native polyacrylamide gel as described [Benezra, 1994 #463].

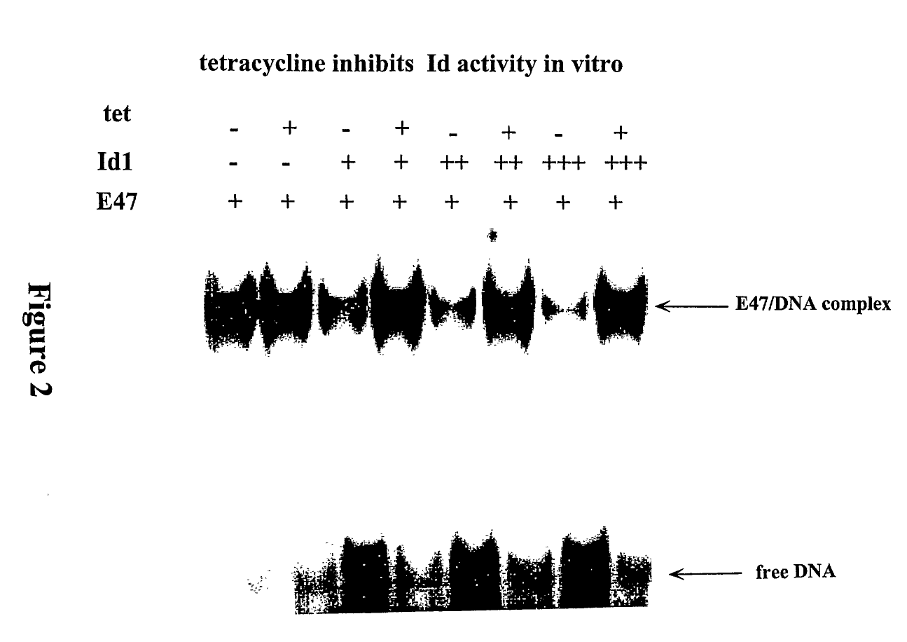

In this experiment, 50 nanograms of E47 protein (described in Example I) was present in reaction mixtures of 100, 200, and 400 nanograms of Id1, designated +, ++, and +++ respectively in FIG. 2. Tetracycline was added to the reactions as indicated (+) to about 200 Tg/ml final concentration. Radiolabeled DNA to which E47 binds was then introduced in each mixture. These mixtures were permitted to Each of the mixtures was permitted to incubate at room temperature for about 30 minutes. Then, the mixtures were electrophoresed in an 8% native polyacrylamide gel as described in Benezra, 1994.

After electrophoresing the samples, the gel was dried and exposed to autoradiographic film to make an autoradiograph of the gel. A photograph of the autoradiograph is set forth in FIG. 2.

Results

As explained above, E47 binds to the radiolabeled DNA, forming an E47/DNA complex. In lanes 1 and 2 of FIG. 2, wherein the only protein in the sample was E47, an E47/DNA complex band was formed, and very little free DNA remained. However when both E47 and Id1 were present with the radiolabeled DNA, a substantial amount of the E47 protein dimerized with Id1. Thus, very little E47 was available to form a complex with the radiolabeled DNA. This result can readily be seen in lanes 3, 5, and 7 of FIG. 2. In fact, as the concentration of Id1 increased, a sufficient amount was available to dimerize with nearly all E47 in the samples. Thus, the E47/DNA complex band decreased in intensity from lane 3, to lane 5, to lane 7 of FIG. 2.

However, in Lanes 4, 6 and 8, tetracycline was present. Lane 4 has the identical amount of E47 and Id1 present as is present in lane 3. However, the E47/DNA complex band in lane 4 is much more intense than the E47/DNA complex band of Lane 3. Thus, the tetracycline present in the sample in lane 4 interacted with Id and prevented its dimerization to E47. As a result, the amount of free E47 increased, which permitted increased formation of E47/DNA complex. The same can be said for lane 6 with respect to lane 5, and lane 8 with respect to lane 7. Hence, tetracycline clearly inhibits Id activity, i.e., dimerizing with a protein member of the HLH super family, in vitro.

### Example III

### Tetracycline Inhibits the Activity of Id1 in Tissue Culture Models

General Strategy:

Plasmids capable of expressing proteins when introduced into mammalian culture cells were used to test the activity of those proteins. In the assay depicted below, a plasmid that contains sequences to which the E12 protein binds upstream of a reporter gene (4R-TK-CAT) ix mixed with an E12 encoding plasmid and an Id plasmid to see if Id can inhibit the ability of E12 protein to bind and activate the reporter gene, in a similar to that described in Pesce, 1993 #163. If E12 binds to its sequence in the 4R-TK-CAT vector, it causes expression of the chloramphenical acetyltransferase (CAT) gene. The protein product of the CAT gene has an enzymatic activity that can be quantified. Uptake of the various plasmids was normalized to the expression of luciferase activity from a separate plasmid added in equal amounts to all cultures.

In the experiment depicted, E12 plasmid causes the activation of 4R-TK-CAT in the presence or absence of tetracycline (100 Tg/ml), demonstrating that E12 is not effected by tetracycline. This result corresponds to the results set forth in Examples I and II above. However, when Id1 plasmid was added at a 3:1 molar excess of E12, i.e., Id1 (low), E12 binding activity was lost in the absence of tetracycline, but partially restored in the presence of tetracycline. Consequently, tetracycline inhibited the activity of Idl in a cell. When higher levels of the Idl gene are expressed (Idl high, 10:1 molar excess over E12), tetracycline is no longer effective due to titration.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

It is further to be understood that all base sizes or amino acid sizes, and all molecular weight or molecular mass values, given for nucleic acids or polypeptides are approximate, and are provided for description.

Various publications are cited herein, the disclosures of which are incorporated by reference in their entireties.

What is claimed is:

1. A method of modulating angiogenesis in an animal comprising the administration of an effective amount of a compound that modulates dimerization between an Id protein and a protein member of the HLH superfamily.

2. The method of claim 1, wherein the compound interacts with the Id protein and modulates its dimerization with a protein of the HLH superfamily.

3. The method of claim 2, wherein modulating angiogenesis comprises inhibiting angiogenesis, and the compound's interaction with the Id protein inhibits dimerization of the Id protein with a protein of the HLH superfamily.

4. The method of claim 3, wherein the compound comprises antibody having the Id protein as an immunogen, or an analog or derivative of tetracycline.

5. The method of claim 5, wherein the analog or derivative of tetracycline has a general structure comprising: 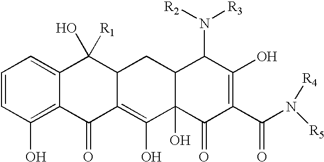
wherein R₁, R₂, R₃, R₄, and R₅ may be the same or different, and comprise H, lower alkyl (C₁-C₄), C₁-C₄ alkoxyl, cycloalkyl, aryl, or heterocyclic ring structures.

6. The method of claim 1, wherein the Id protein comprises Id1, Id2, Id3, or Id4.

7. The method of claim 1, wherein modulating angiogenesis comprises inhibiting angiogenesis, and the compound inhibits expression of an Id gene.

8. The method of claim 7, wherein the compound is an antisense compound comprising at least one phosphodiester analog bond, wherein said antisense compound inhibits translation of RNA produced from transcription of the Id gene.

9. The method of claim 8, wherein the Id gene comprises Id1, Id2, Id3, or Id4.

10. A method of modulating tumor growth and metastasis in an animal comprising the administration of an effective amount of a compound that interacts with an Id protein, and modulates dimerization of the Id protein with a protein member of the HLH superfamily.

11. The method of claim 10, wherein modulating tumor growth and metastasis comprises inhibiting tumor growth and metastasis, and modulating dimerization comprises inhibiting dimerization.

12. The method of claim 11, wherein the compound comprises an antibody to the Id protein, or analog of tetracycline, or a derivative of tetracycline.

13. The method of claim 12, wherein the analog or derivative of tetracycline has a general structure comprising: 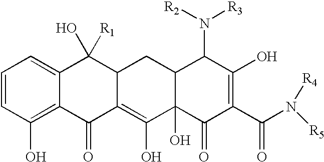
wherein R₁, R₂, R₃, R₄, and R₅ may be the same or different, and comprise H, lower alkyl (C₁-C₄), C₁-C₄ alkoxyl, cycloalkyl, aryl, or heterocyclic ring structures.

14. The method of claim 11, wherein inhibiting tumor growth and metastasis comprises preventing development of tumors and metastasis in the animal after administration of an effective amount of the compound.

15. The method of claim 14, wherein the compound comprises an antibody having the Id protein as an immunogen, an analog of tetracycline, or a derivative of tetracycline.

16. The method of claim 15, wherein the analog or derivative of tetracycline has a general structure comprising: 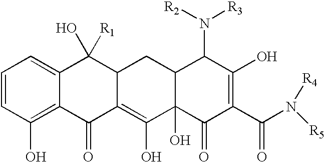
wherein R₁, R₂, R₃, R₄, and R₅ may be the same or different, and comprise H, lower alkyl (C₁-C₄), C₁-C₄ alkoxyl, cycloalkyl, aryl, or heterocyclic ring structures.

17. The method of claim 10, wherein modulating tumor growth and metastasis comprises shrinling tumors present in the animal prior to administration of the effective amount of the compound.

18. The method of claim 17, wherein the compound comprises an antibody having the Id protein as an immunogen, an analog of tetracycline, or a derivative of tetracycline.

19. The method of claim 19, wherein the analog or derivative of tetracycline has a general structure comprising: 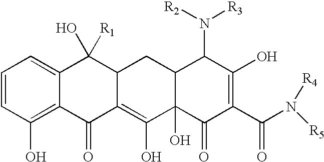
wherein R₁, R₂, R₃, R₄, and R₅ may be the same or different, and comprise H, lower alkyl (C₁-C₄), C₁-C₄ alkoxyl, cycloalkyl, aryl, or heterocyclic ring structures.

20. The method of claim 10, wherein modulating tumor growth and metastasis comprises preventing tumor growth and metastasis in the animal, wherein the animal is free of tumors or metastasis prior to administration of the effective amount of the compound.

21. The method of claim 20, wherein the compound comprises an antibody having the Id protein as an immunogen, an analog of tetracycline, or a derivative of tetracycline.

22. The method of claim 21, herein the analog or derivative of tetracycline has a general structure comprising: 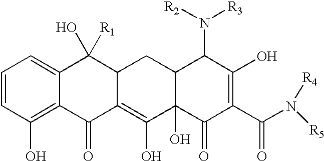
wherein R₁, R₂, R₃, R₄, and R₅ may be the same or different, and comprise H, lower alkyl (C₁-C₄), C₁-C₄ alkoxyl, cycloalkyl, aryl, or heterocyclic ring structures.

23. The method of claim 10 wherein the Id protein comprises Id1, Id2, or Id3.

24. A method for screening potential drugs or agents which modulate angiogenesis, comprising the steps of:
a) providing an Id protein;
b) contacting the potential drug or agent to the Id protein; and
c) determining whether the potential drug or agent is bound to the Id protein, wherein binding of the potential drug or agent to the Id protein is may be indicative of the ability of the drug or agent to modulate angiogenesis.

25. The method of claim 24, further comprising the steps of conjugating the Id protein to a solid phase resin prior to contacting the potential drug or agent to the Id protein, and removing the Id protein from the solid phase resin prior to determining whether the potential drug or agent is bound to the Id protein.

26. The method of claim 25, wherein the conjugating step comprises binding the Id protein to a cobalt resin at protein to resin ratio that allows for saturation of the resin with the Id protein.

27. The method of claim 25, wherein the removing step comprises contacting the Id protein conjugated to the solid phase resin to an imidazole solution.

28. The method of claim 24, wherein the Id protein comprises Id1, Id2, Id3, or Id4.

29. The method of claim 24, wherein the potential drug or agent is a member of a library of compounds, and the contacting step comprises contacting the library of compounds to the Id protein.

30. The method of claim 29, wherein the library of compounds comprises a mixture of compounds or a combinatorial library.

31. The method of claim 29, wherein the library of compounds comprises analogs or derivatives of tetracycline.

32. A method of screening for compounds which selectively bind to an Id protein comprising:
(a) complexing a Id protein to a solid support;
(b) contacting the complexed protein/solid support with an aqueous solution comprising a compound that is being screened for the ability to selectively bind to the Id protein; and
(c) determining whether the compound selectively binds to the Id protein such that the Id protein is prevented from dimerizing with a protein member of the HLH superfamily.

33. The method of claim 32, wherein the solid support is selected from the group comprising: cobalt, insoluble polystyrene beads, PVDF, and polyethylene glycol.
